(19) United States
(12) Patent Application Publication
(10) Pub. No.: US 2015/0088038 A1
(43) Pub. Date: Mar. 26, 2015

(54) STANDUP ASSISTANCE APPARATUS AND METHOD

(71) Applicant: KABUSHIKI KAISHA TOSHIBA, Tokyo (JP)

(72) Inventors: Izumi Fukunaga, Tokyo (JP); Takamitsu Sunaoshi, Yokohama (JP); Hideki Nukada, Yokohama (JP)

(21) Appl. No.: 14/489,578

(22) Filed: Sep. 18, 2014

(30) Foreign Application Priority Data
Sep. 20, 2013 (JP) 2013-196196

(51) Int. Cl. A61H 1/00; A61B 5/11; A61B 5/00; G01P 15/00

(52) U.S. Cl. CPC A61H 1/005; G01P 15/00; A61B 5/1116; A61B 5/486; A61B 5/7264; A61B 5/4836; A61H 2201/1633; A61H 2201/5007; A61H 2201/5084; A61H 2205/086; A61H 2230/625
USPC 600/595; 601/26

(57) ABSTRACT

According to one embodiment, a standup assistance apparatus includes a measurement unit, a detection unit and a determination unit. The measurement unit is configured to measure a center-of-gravity acceleration at which a position of a center-of-gravity of a subject moves. The detection unit is configured to detect whether or not buttocks of the subject contact a surface. The determination unit is configured to determine a standup ability of the subject in accordance with whether or not the buttocks contact the surface when the center-of-gravity acceleration reaches a first extreme value or a second extreme value which is a next extreme value of the first extreme value.

### CROSS-REFERENCE TO RELATED APPLICATIONS

This application is based upon and claims the benefit of priority from Japanese Patent Application No. 2013-196196, filed Sep. 20, 2013, the entire contents of which are incorporated herein by reference.

### FIELD

Embodiments described herein relate generally to a standup assistance apparatus and method.

### BACKGROUND

Any person unable to stand up because of disease or weakened muscles needs some assistance to move. A technique for assisting such a person to stand up, for example, is available. This technique uses a load sensor embedded in the surface of a seat. When a user sitting on the seat moves forward to stand up, the seat is controlled in accordance with the output of the load sensor, whereby the seat surface moves up and forward, assisting the user to stand up.

### BRIEF DESCRIPTION OF THE DRAWINGS

FIG. 1 is a block diagram illustrating a standup assistance apparatus according to a first embodiment;

FIG. 2 is a flowchart illustrating how a standup ability determination unit operates in the standup assistance apparatus according to the first embodiment;

FIG. 3 is a diagram illustrating a first example in which a total floor counter-force and a seat-surface counter-force change as a subject stands up;

FIG. 4 is a diagram illustrating a second example in which the total floor counter-force and the seat-surface counter-force change as the subject stands up;

FIG. 5 is a flowchart illustrating how the standup ability determination unit operates in a modification of the first embodiment;

FIG. 6 is a diagram illustrating a first example in which the total floor counter-force and the seat-surface counter-force change as the subject stands up, assisted by the modification of the first embodiment;

FIG. 7 is a diagram illustrating a second example in which the total floor counter-force and the seat-surface counter-force change as the subject stands up, assisted by the modification of the first embodiment;

FIG. 8 is a diagram illustrating an example in which the standup ability of the subject is determined from the acceleration of the subject;

FIG. 9 is a block diagram illustrating a standup assistance apparatus according to a second embodiment;

FIGS. 10A and 10B are diagrams illustrating examples of how the standup assistance apparatus according to the second embodiment is used;

FIG. 11 is a diagram illustrating a separate example of the assistance output unit of the standup assistance apparatus according to the second embodiment; and

FIG. 12 is a flowchart illustrating how the standup assistance apparatus according to the second embodiment operates.

### DETAILED DESCRIPTION

In the method described above, it is detected when the occupant starts moving, but no determination is made of whether the occupant is able to stand up or not, and the standup-assistance ability of the apparatus is not automatically adjusted in accordance with the muscle power of the occupant. That is, the apparatus always raises up the seat surface with the same power, irrespective of the user's physical ability (hereinafter referred to as “standup ability”), including muscle power and balancing ability. The apparatus is not designed to utilize the standup ability the occupant possesses and thereby prevent their ability from decreasing from the present level.

In general, according to one embodiment, a standup assistance apparatus includes a measurement unit, a detection unit and a determination unit. The measurement unit is configured to measure a center-of-gravity acceleration at which a position of a center-of-gravity of a subject moves. The detection unit is configured to detect whether or not buttocks of the subject contact a surface. The determination unit is configured to determine a standup ability of the subject in accordance with whether or not the buttocks contact the surface when the center-of-gravity acceleration reaches a first extreme value or a second extreme value which is a next extreme value of the first extreme value.

In the following, the standup assistance apparatus and method according to an embodiment of the present disclosure will be explained with reference to the drawings. In the following embodiments, the explanation of the elements with the same reference numerals will be omitted for brevity as their operations will be the same.

### First Embodiment

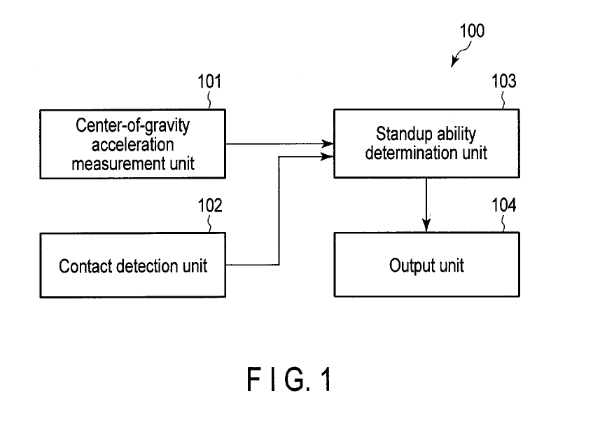

A standup assistance apparatus according to the first embodiment will be described with reference to the block diagram of FIG. 1.

The standup assistance apparatus 100 according to the first embodiment includes a center-of-gravity acceleration measurement unit 101, a contact detection unit 102, a standup-ability determination unit 103, and an output unit 104.

The center-of-gravity acceleration measurement unit 101 detects the center-of-gravity acceleration of the subject. The subject is a user unable to stand up by themselves, and therefore needs to use the apparatus for rehabilitation. The center-of-gravity acceleration is the acceleration at which the center-of-gravity of the subject moves, for example, in the vertical direction. The center-of-gravity acceleration may be measured by, for example, an acceleration sensor, an image sensor, motion capture, a force sensor, or a weight sensor.

The center-of-gravity acceleration may be calculated as follows. An acceleration sensor is attached to the trunk of the subject and measures the acceleration, while a geomagnetism sensor is used in determining the vertical direction, enabling the center-of-gravity acceleration of the subject to be calculated. If an image sensor or motion capture is used, the center-of-gravity can be calculated from the positions of the subject's joints, thereby determining the center-of-gravity acceleration. If a weight sensor is used, the center-of-gravity acceleration can be determined from the floor counter-force, which is proportional to the center-of-gravity acceleration. In this embodiment, a weight sensor is embedded in the floor that supports the subject, and shall hereinafter be called a “floor-surface weight sensor.” Hence, the total floor counter-force measured by the floor-surface weight sensor is used as a physical quantity corresponding to the center-of-gravity acceleration.

The contact detection unit 102 detects whether or not the subject's buttocks are in contact with the surface of the seat to determine the contact status of the subject. The surface is, for example, the seat surface the subject contacts while occupying the seat. This embodiment is based on the assumption that the subject stands up from the seat. The weight sensor is therefore embedded in the seat surface (referred to as a floor-surface weight sensor). Nonetheless, if the subject sits on the floor, the weight sensor is also embedded in that part of the floor which the subject's buttocks may contact. In this case, too, the center-of-gravity acceleration can be measured in the same way. The buttocks are determined to not be contacting the seat surface if the weight sensor detects 0 kgf, and to be contacting the seat surface if the weight sensor detects a force greater than 0 kgf. The contact status determined from the output of the weight sensor shall be called a “seat-surface counter-force.” The seat-surface counter-force may be the weight measurement obtained by the floor-surface weight sensor, or may be represented by a binary value showing whether or not the subject's buttocks are in contact with the seat surface.

Whether or not the subject's buttocks are in contact with the seat surface may be determined not only by the weight sensor, but may also be determined by, for example, at least one sensor selected from the group consisting of a contact sensor, an image sensor, motion capture, a temperature sensor, a strain sensor, an infrared beam sensor, and a laser range finder. Specifically, if one contact sensor is used, it can be determined whether or not the buttocks are in contact with the seat surface. If two or more contact sensors are used, it can be detected whether or not the buttocks contact a specific part of the seat surface. If an image sensor, motion capture, and infrared beam sensor are used, both the buttocks and the seat surface are detected, and whether or not the buttocks are in contact with the seat surface is determined from the distance between the two. If a temperature sensor is embedded in the seat surface, it can determine that the buttocks contact the seat surface if the temperature the sensor detects is equal to or greater than a threshold value. If a strain sensor is embedded in the seat surface, it can determined that the buttocks contact the seat surface if the strain the sensor detects is equal to or greater than a threshold value. Alternatively, a laser range finder may be positioned to detect the distance between the seat surface and the buttocks contacting the seat surface. The change in distance can be detected as the subject rises from the seat.

The standup-ability determination unit 103 receives center-of-gravity acceleration data and contact status data from the center-of-gravity acceleration measurement unit 101 and the contact detection unit 102, respectively. The standup-ability determination unit 103 determines the subject's ability to stand up, i.e., their physical ability including muscle power and balancing ability, in accordance with whether or not the subject's buttocks contact the seat surface at the time the center-of-gravity acceleration reaches a first extreme value, and also at the time the center-of-gravity acceleration reaches a second extreme value. In this embodiment, the first and second extreme values are, respectively, the maximum value and minimum value the center-of-gravity acceleration has as the total floor counter-force (i.e., center-of-gravity acceleration), if the center-of-gravity acceleration is regarded as increasing upward in the vertical direction. Also, in this embodiment the standup ability is determined in three or more levels, from the total floor counter-force and the seat-surface counter-force. The following description is based on the assumption that the lower the value of the standup ability level is, the higher the subject's standup ability, and that the higher the value of the standup ability level is, the lower the subject's standup ability.

The output unit 104 receives the determination result of the subject's standup ability from the standup-ability determination unit 103, and outputs the determination result. That is, the output unit 104 is, for example, a display showing the data representing the subject's standup ability. The subject's standup ability displayed includes, for example, the standup ability level and the index based on the standup ability level. The output unit 104 may output the center-of-gravity acceleration data (change over time), in addition to the data representing the standup ability.

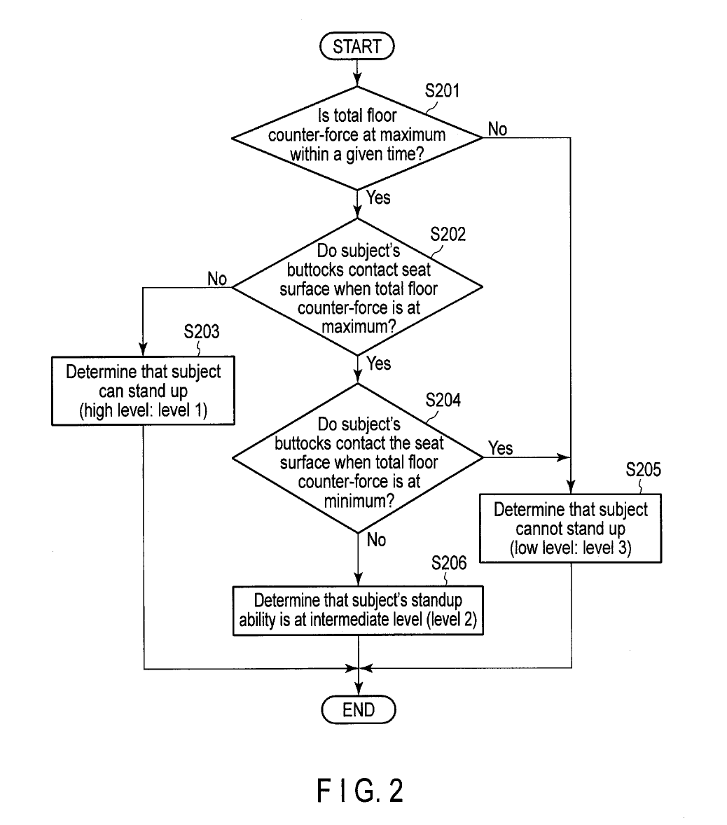

Next, the standup-ability determination unit 103 will be explained with reference to the flowchart of FIG. 2. The standup-ability determination unit 103 may acquire, at regular intervals, the time-series data of the total floor counter-force from the center-of-gravity acceleration measurement unit 101 and the seat-surface counter-force from the contact detection unit 102, thereby determining the subject's standup ability. In order to save power consumption, the standup-ability determination unit 103 may also start operating when the user pushes a start button, or when the total floor counter-force or the seat-surface counter-force changes to a threshold value or a greater value.

In Step S201, it is determined whether or not a maximum total floor counter-force value is present within a given time from the start of the process of detecting the center-of-gravity acceleration. To determine this, it suffices to detect the change in the total floor counter-force, distinguished from noise. If the maximum total floor counter-force value is present in the given time, the process goes to Step S202. If the maximum total floor counter-force value is not present in the given time, the process goes to Step S205.

In Step S202, it is determined whether or not the subject's buttocks contact the seat surface at the time (referred to as a first timing) when the total floor counter-force reaches the maximum value. If the subject's buttocks contact the seat surface, the process goes to Step S204. If the subject's buttocks do not contact the seat surface, the process goes to Step S203.

In Step S203, it is determined that the subject can stand up by themselves. The subject's standup ability is therefore determined to be at “high level (level 1)”.

In Step S204, it is determined whether or not the subject's buttocks contact the seat surface at the time (also called “second timing”) the total floor counter-force takes the minimum value. If the subject's buttocks contact the seat surface, the process goes to Step 205. If the subject's buttocks contact the seat surface, the process goes to Step 206.

In Step S205, it is determined that the subject is unable to stand up by themselves. The subject's standup ability is therefore determined to be at “low level (level 3)”.

In Step S206, it is determined that the subject's buttocks have left the seat surface at least once. The subject is therefore considered able to stand up, but not so well. The subject's standup ability is therefore determined to be at “intermediate level (level 2)”. The standup-ability determination unit 103 finishes a determination process.

In case it is difficult to determine the maximum and minimum values of the total floor counter-force, a moving average or a filter may be used to remove the noise, and the maximum or minimum value may then be determined. If a change greater than the noise is observed, both the maximum value and the minimum value may be determined.

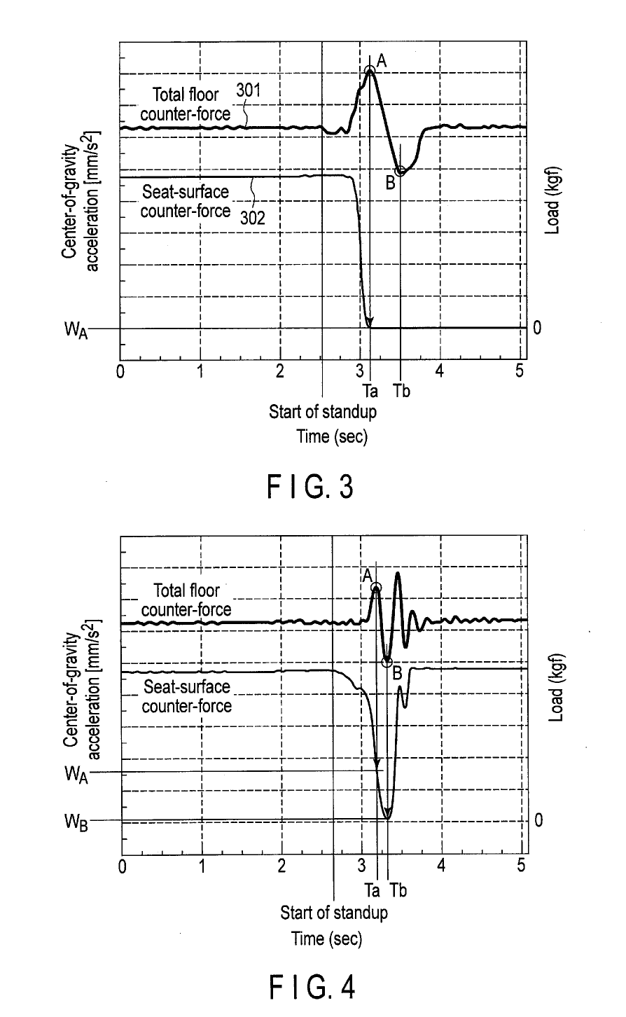

Next, the operation of standup-ability determination unit 103 will be explained in greater detail with reference to FIGS. 3 and 4.

FIG. 3 is a diagram showing a first example in which the total floor counter-force and the seat-surface counter-force change over time as the occupant stands up. In FIG. 3, the vertical axis is the center-of-gravity acceleration [mm/s²] and is plotted on the vertical axis, and the time [s] is plotted on the horizontal axis. In FIG. 3, the upper line 301 shows how the total floor counter-force changes over time, and the lower line 302 shows how the seat-surface counter-force changes over time.

In FIG. 3, a time point “standup start” is the time the user pushes the start button, or the time the center-of-gravity acceleration decreases as the subject shifts their body before standing up. This decrease in center-of-gravity acceleration can be detected as a change in center-of-gravity acceleration which is not less than the change resulting from noise and is not more than the threshold value for determining the maximum or minimum value.

The standup-ability determination unit 103 detects the maximum and minimum values of the total floor counter-force at time after the time point “standup start.”

In FIG. 3, point A indicates the maximum value, and Ta indicates the time at which the total floor counter-force reaches the maximum value A. After reaching the maximum value A, the slowing acceleration of the subject to stop their movement causes the total-floor counter-force to acquire the minimum value. In FIG. 3, point B indicates the minimum value, Tb indicates the time the total floor counter-force takes the minimum value B.

If the subject has sufficient ability to stand up as an able-bodied person does, the seat-surface counter-force 302 will decrease to zero at time Ta and time Tb. This shows that the subject's buttocks have left the seat surface, or that the subject has stood up already. The subject's standup ability is therefore determined to be at “high level (level 1)”.

FIG. 4 is a diagram showing a second example in which the total floor counter-force and the seat-surface counter-force change over time as the subject cannot stand up.

As shown in FIG. 4, the seat-surface counter-force does not decrease to zero at time Ta when the total floor counter-force has the maximum value or at time Tb when the total floor counter-force has the minimum value. That is, the subject's buttocks have not left the seat surface (the subject remains seated), or the maximum value when the subject is about to stand up is buried in the noise and is unable to be detected. In this case, the subject's standup ability is determined to be at “low level (level 3)”.

Any data change not pertaining to either the data waveform of FIG. 3 or the data waveform of FIG. 4 may be considered as representing standup ability of the intermediate level (level 2).

### Modification of the First Embodiment

The intermediate level may be classified into sub-levels in a modification of the first embodiment.

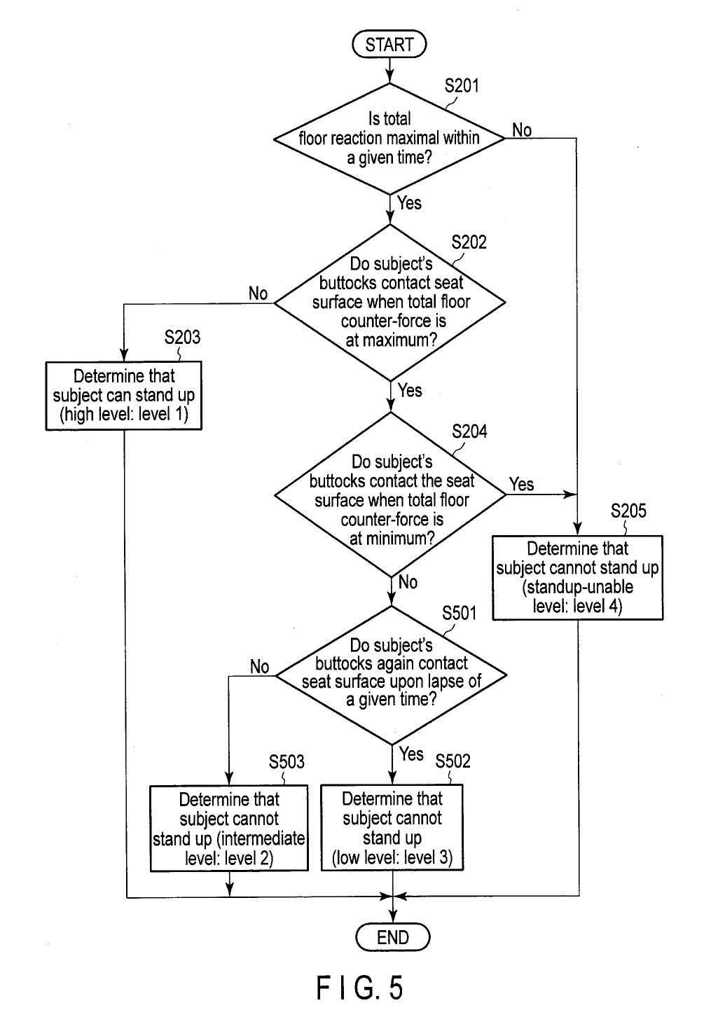

The operation of standup-ability determination unit 103 in a modification of the first embodiment will be explained with reference to the flowchart of FIG. 5. In the modification, the intermediate level is classified into two sub-levels. As a result, it is determined whether or not the subject's standup ability is at one of four levels. In the modification, Steps S201 to S205 are identical to those shown in FIG. 2, and will not be described. For convenience, the subject's standup ability level determined in Step S205 will be called “standup-unable level (level 4)”.

In Step S501, it is determined whether or not the subject's buttocks again contact the seat surface at a time elapsed a given time from the time the total floor counter-force reaches the minimum value. If the buttocks contact the seat surface again, the process goes to Step S502. If the buttocks do not contact the seat surface again, the process goes to Step S503. The given time is preferably 500 ms or less, but it is not limited to this and it may have any appropriate value.

In Step S502, it is determined that the buttocks have at least partially left the seat surface. This shows that the subject has some ability to stand up. The subject's standup ability is therefore determined to be at “low level (level 3),” which is higher than level 4.

In Step S503, it is determined that the subject cannot rise from the seat surface at the time the total floor counter-force is at maximum, but can rise, with the subject's buttocks finally leaving the seat surface. Namely, the subject is found at “intermediate level (level 2),” and is able to stand up, but needs more time to rise than at high level (level 1).

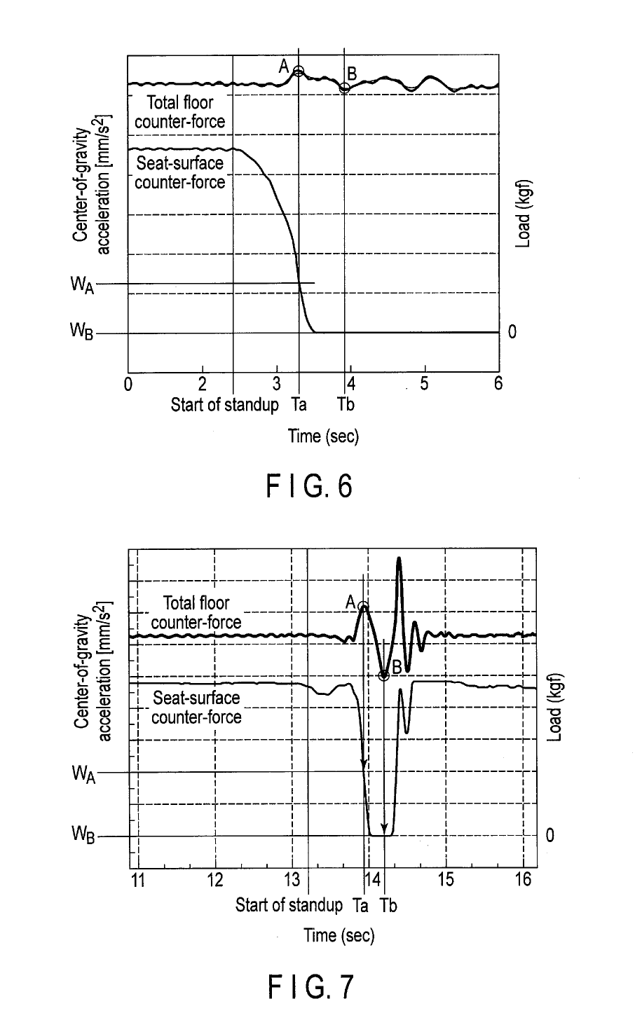

Some specific examples of modifications of the first embodiment will be described with reference to FIG. 6 and FIG. 7.

FIG. 6 is a diagram showing how the total floor counter-force and the seat-surface counter-force change as a subject with a standup ability at the intermediate level (level 2) stands up, assisted by a modification of the first embodiment.

As shown in FIG. 6, at time Ta when the total floor counter-force has the maximum value A, the seat-surface counter-force is not zero. At time Tb when the total floor counter-force has the minimum value B, the seat-surface counter-force is zero and remains zero afterward. In this case, the subject slowly stands up, and their standup ability is considered lower than high level (level 1) at which the subject can stand up quickly. The subject is therefore considered having standup ability at intermediate level (level 2).

FIG. 7 is a diagram showing how the total-floor counter-force and the seat-surface counter-force change as the subject of standup ability at low level (level 3) stands up, assisted by the modification of the first embodiment.

As shown in FIG. 7, at time Ta when the total floor counter-force has the maximum value A, the seat-surface counter-force is not zero. At time Tb when the total floor counter-force has the minimum value B, the seat-surface counter-force is zero and remains zero afterward. Upon elapsing a given time, the seat-surface counter-force starts increasing. This can be thought of as a case where the subject rises a little but sits down again. Hence, the ability of the subject to stand is found at low level (level 3), which is higher than standup-unable level (level 4).

In the example described with reference to FIG. 2 to FIG. 7, the total-floor counter-force is regarded as increasing from minimum value to maximum value, upward in the vertical direction, and may be regarded as increasing downward in the vertical direction. If this is the case, the maximum value and the minimum value replace each other, but the total-floor counter-force can be determined in the same way.

As described in the example above, the subject's standup ability is determined from their center-of-gravity acceleration in the vertical direction. However, the subject's standup ability can also be determined from the amplitude of their acceleration. The amplitude of acceleration may be the sum of the X-axis vector component (in the subject's left-right direction), Y-axis vector component (in the subject's fore-aft direction) and Z-axis vector component (in the vertical direction), or may be the Z-axis vector component only. If the subject's standup ability is determined from the amplitude of their acceleration, it may be determined at the time it reaches a first extreme value and at the time it reaches a second extreme value. The first and second extreme values reach maximum if they increase upward in the vertical direction, and reach minimum if they increase downward in the vertical direction.

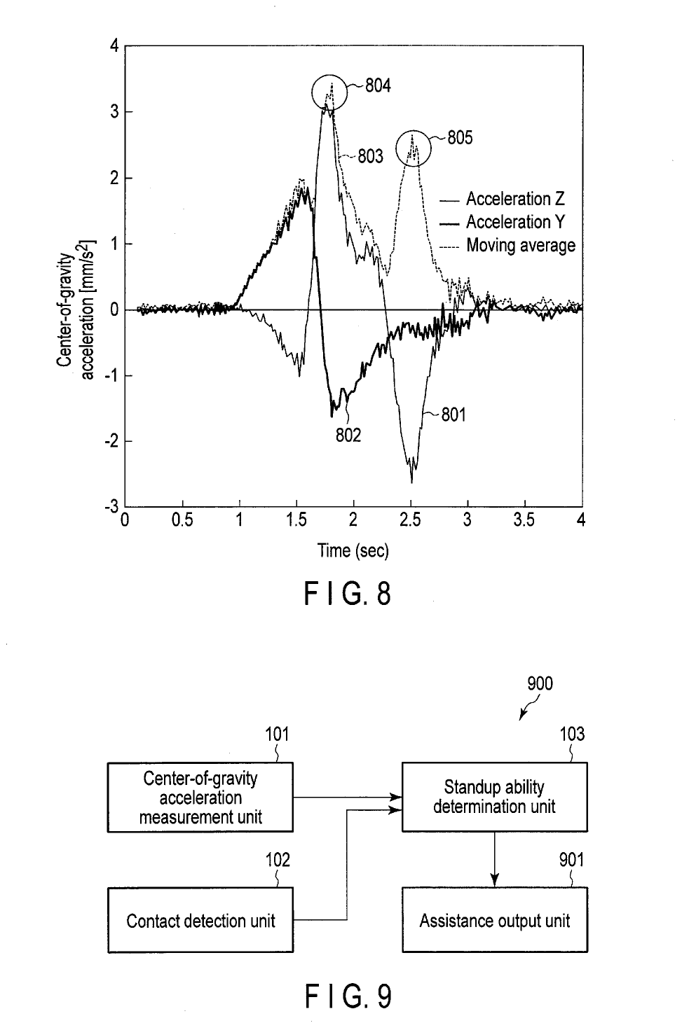

An example in which the subject's standup ability is determined from the acceleration of the subject will be described with reference to FIG. 8.

FIG. 8 shows how the subject's acceleration changes in the case where the subject's standup ability is determined at high level (level 1). The center-of-gravity acceleration is plotted on the vertical axis, and the time is plotted on the horizontal axis. In FIG. 8, lines 801 and 802 show how the acceleration in Z axis and the acceleration in Y axis change over time, respectively, and line 803 shows how the vector of the X-, Y-, and Z-axis acceleration components changes over time. As seen from line 803, the Z-axis (vertical direction) component is predominant while the subject is standing up. The three-component vector (i.e., X-, Y-, and Z-axis acceleration components) changes in a way similar to the way the vertical-direction component changes. Therefore, extreme value 804 is observed as a first extreme value. If extreme value 805 is then observed as a second extreme value, it will be determined that the subject has stood up quickly, and the subject's standup ability is determined to be at “high level (level 1).

According to the embodiments described above, the first embodiment can accurately determine the subject's standup ability from the change in their center-of-gravity acceleration. That is, the standup ability the subject has at any time in any physical state can be determined. Since the subject's center-of-gravity acceleration may be measured by, for example, a weight sensor, many users can use the standup assistance apparatus without the need to set parameters prior to using it.

### Second Embodiment

The second embodiment differs from the first embodiment in that it uses an assistance output unit to help the subject to stand up in accordance with the subject's determined standup ability.

A standup assistance apparatus according to the second embodiment will be described with reference to the block diagram of FIG. 9.

The standup assistance apparatus 900 according to the second embodiment includes a center-of-gravity-acceleration measurement unit 101, a contact detection unit 102, a standup-ability determination unit 103, and an assistance output unit 901. The standup assistance apparatus 900 is identical to the standup assistance apparatus 100 according to the first embodiment, except for the assistance output unit 901. Therefore, the units 101, 102, and 103 will not be described again.

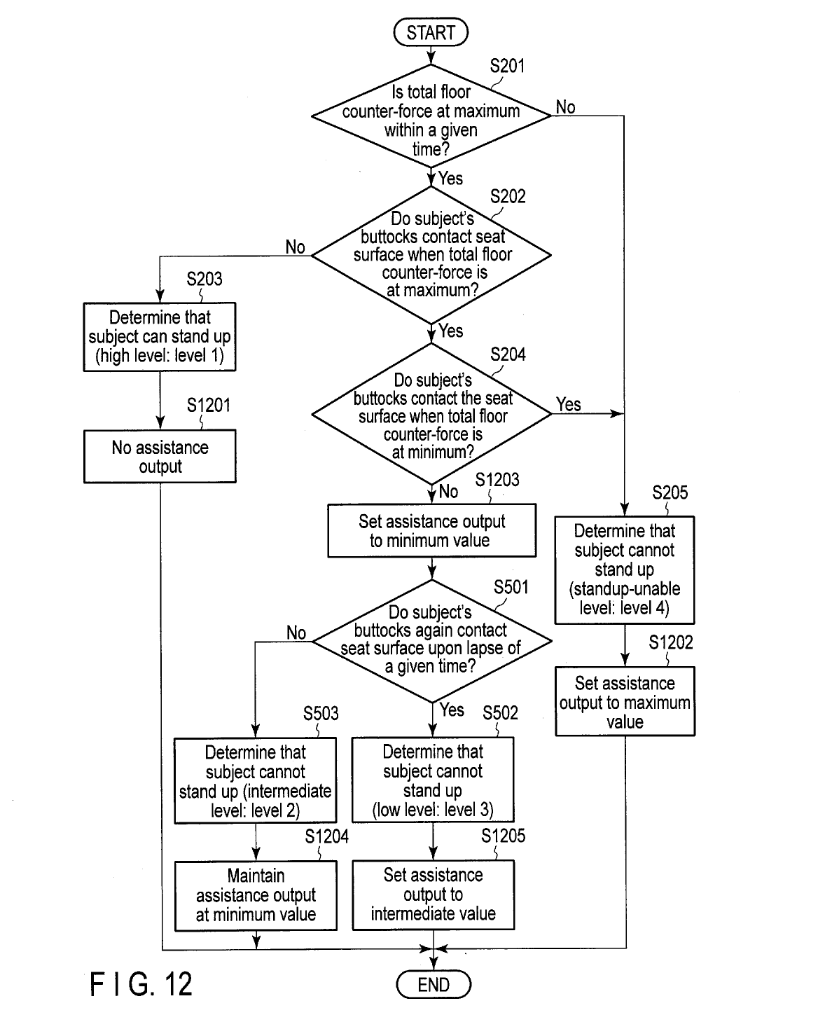

The assistance output unit 901 receives the determination result of the subject's standup ability from the standup-ability determination unit 103, and assists the subject in accordance with the determination result. The lower the standup ability of the subject, the more assistance the subject needs to stand up. Therefore, the standup-ability determination unit 103 generates a physical output inversely proportional to the subject's standup ability, in order to help the subject to stand up. The assistance output unit 901 may include a motor. In this case, the motor torque is increased in inverse proportion to the standup ability, generating a larger physical output. The method of outputting the physical output will be described later with reference to FIG. 12.

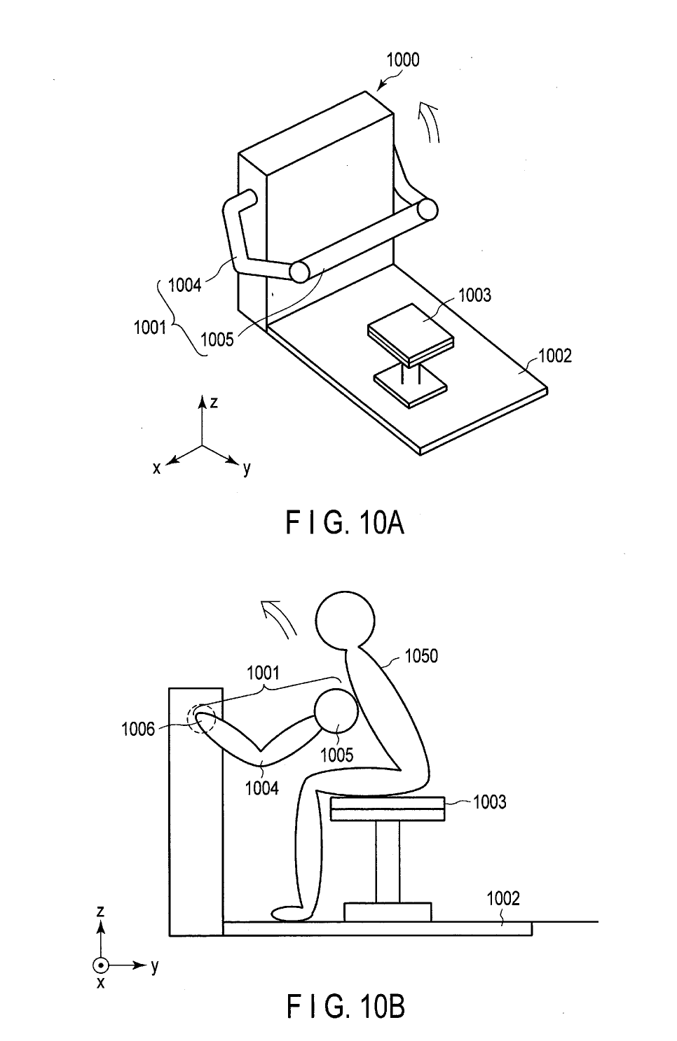

An example of using the standup assistance apparatus according to the second embodiment will be described with reference to FIGS. 10A and 10B.

FIGS. 10A and 10B show a standup assistance apparatus 1000 according to the second embodiment. This apparatus 1000 includes an assistance output unit 1001, a floor 1002, and a chair 1003.

The assistance output unit 1001 includes arms 1004 and a handle 1005. The handle 1005 is connected, at both ends, to the arms 1004. The arms 1004 are rotated with a force inversely proportional to the subject's determined standup ability. A floor weight sensor is embedded in the floor 1002 to detect the total floor counter-force. A chair weight sensor is embedded in the chair 1003 to detect the seat-surface counter-force.

As shown in FIG. 10B, the subject 1050 may sit on the chair 1003 and may then hold the handle 1005 to stand up from the chair 1003.

In this case, the arms 1004 are rotated around an axle 1006 in the direction of the arrow (in a counterclockwise direction), with the force set in accordance with the standup ability determined by the standup assistance apparatus 1000. So rotated, the arms 1004 help the subject to stand up because the subject keeps holding the handle 1005.

The arms 1004 may be moved up in the vertical direction, not rotated in the direction of the arrow. In this case, too, the standup assistance apparatus 1000 can help the subject to stand up.

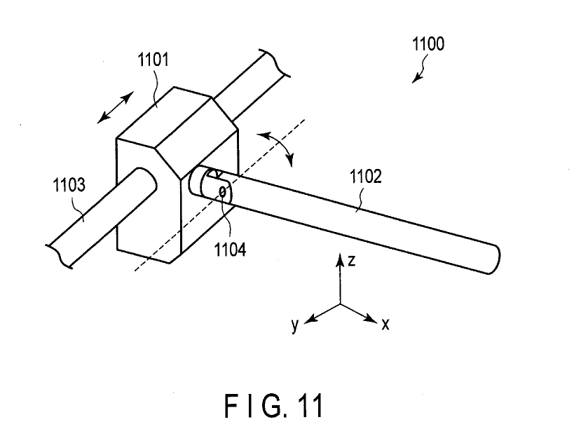

FIG. 11 shows another type of an assistance output unit for use in the standup assistance apparatus 1000. The assistance output unit 1100 shown in FIG. 11 includes a moving unit 1101 and a handle 1102.

The moving unit 1101 is mounted on a fixed bar 1103 and can slide on the fixed bar 1103.

The handle 1102 is connected to the moving unit 1101 and is located above the knee joints of the subject sitting on the chair 1003 (FIG. 10). The handle 1102 can be rotated in the z direction, around a pin 1104, so that the assistance output unit 1100 may be stored in a confined space.

To help the subject to stand up, the fixed bar 1103, for example, is inclined in the y-z plane, not parallel to the y-axis. This enables the moving unit 1101 to move in both the y-axis direction and the z-axis direction. The assistance output unit 1100 can therefore help the subject to stand up. The fixed bar 1103 may be arranged parallel to the y-axis. In this case, a mechanism for moving the moving unit 1101 in the z-axis direction is used to move the unit 1101 in both the y-axis direction and the z-axis direction.

The standup assistance apparatus 1000 shown in FIG. 10 is designed to assist the subject by using the arms extending from the main unit. However, the apparatus may also have an assistance output unit installed on the floor or the wall, an assistance output unit of moving type, or an assistance output unit attached to the subject. Furthermore, the assistance output unit is not limited to the type having arms and a handle, it may also be designed to support or wrap the body of the subject.

The operation of the standup assistance apparatus 900 according to the second embodiment will be explained with reference to the flowchart of FIG. 12.

Steps S201 to S205 and Steps S501 to S503 are identical to those shown in FIG. 5, and will not be described again.

In Step S1201, the subject's standup ability is determined to be at high level (level 1) in Step S203, and the subject does not need to be assisted. Therefore, the assistance output unit 901 generates no assistance outputs.

In /Step S1202, the subject's standup ability is determined to be at the standup-unable level (level 4) in Step S205, and the subject cannot stand up unassisted. Therefore, the assistance output unit 901 generates a maximum assistance output.

In Step S1203, the assistance output unit 901 generates a small assistance output. This is because the subject cannot be considered as having sufficient standup ability if their buttocks contact the seat surface, and thus the subject is desirable to be assisted a little, regardless of whether or not they can later stand up by themselves.

In Step S1204, the assistance output unit 901 maintains the low assistance output if the subject's standup ability is determine in Step S503 to be at intermediate level (level 2).

In Step S1205, the assistance output unit 901 increases the assistance output to an intermediate value if the subject's standup ability is determined in Step S502 to be low level (level 3) and the subject is considered unable to stand up without assistance. Then, the standup assistance apparatus 900 according to the second embodiment stops its operation.

In accordance with any standup ability level determined, the assistance output may be set to a level lower than the ordinary value. In Step S1204, for example, no assistance output may be output, instead of the low assistance output. In Step S1205, the low assistance output may be output, instead of the intermediate assistance output. This helps the subject to enhance their standup ability through rehabilitation, etc.

According to the second embodiment described above, the assistance output is set to an appropriate value in accordance with the subject's determined standup ability. The second embodiment can therefore help the subject to stand up appropriately. Since the assistance output can be set to a value smaller than the value corresponding to the determined standup ability, the second embodiment can achieve rehabilitation effects, such as an increase in the subject's muscle strength.

The embodiments described above are designed to determine the subject's standup ability, assuming that the subject tries to stand up without touching anything. Nonetheless, these embodiments can be applied to the case where the subject is helped to stand up, while touching a wall, handrails, elbow rests, or the like. In this case, weight sensors may be embedded in the wall, handrails, or the like, and the sum of the outputs of the weight sensors may be used as the subject's center-of-gravity acceleration.

It is not absolutely necessary to assist the subject in real time in accordance with the determined standup ability. Rather, the determined standup ability may be utilized as the result of the rehabilitation conducted.

The flow charts of the embodiments illustrate methods and systems according to the embodiments. It will be understood that each block of the flowchart illustrations, and combinations of blocks in the flowchart illustrations, can be implemented by computer program instructions. These computer program instructions may be loaded onto a computer or other programmable apparatus to produce a machine, such that the instructions which execute on the computer or other programmable apparatus create means for implementing the functions specified in the flowchart block or blocks. These computer program instructions may also be stored in a computer-readable memory that can direct a computer or other programmable apparatus to function in a particular manner, such that the instruction stored in the computer-readable memory produce an article of manufacture including instruction means which implement the function specified in the flowchart block or blocks. The computer program instructions may also be loaded onto a computer or other programmable apparatus to cause a series of operational steps to be performed on the computer or other programmable apparatus to produce a computer programmable apparatus which provides steps for implementing the functions specified in the flowchart block or blocks.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. A standup assistance apparatus, comprising:
a measurement unit configured to measure a center-of-gravity acceleration at which a position of a center-of-gravity of a subject moves;
a detection unit configured to detect whether or not buttocks of the subject contact a surface;
a determination unit configured to determine a standup ability of the subject in accordance with whether or not the buttocks contact the surface when the center-of-gravity acceleration reaches a first extreme value or a second extreme value which is a next extreme value of the first extreme value.

2. The apparatus according to claim 1, wherein the center-of-gravity acceleration is acceleration at which the position of the center-of-gravity moves in a vertical direction, the first extreme value is a maximum value of the center-of-gravity acceleration, and the second extreme value is a minimum value of the center-of-gravity acceleration, if the center-of-gravity acceleration is regarded as increasing upward in the vertical direction.

3. The apparatus according to claim 1, wherein the center-of-gravity acceleration is amplitude of acceleration of the position of the center-of-gravity, and the first extreme value and the second extreme value are maximum values of the center-of-gravity acceleration if the center-of-gravity acceleration is regarded as increasing upward in the vertical direction.

4. The apparatus according to claim 1, wherein the determination unit classifies the standup ability into a first level, a second level and a third level, the first level being a level at which the subject is able to stand up, the second level being a level lower than the first level and higher than a third level, the third level being a level at which the subject is unable to stand up.

5. The apparatus according to claim 4, wherein the determination unit determines that;
the standup ability is at the first level if the buttocks do not contact the surface at a first timing when the center-of-gravity acceleration reaches the first extreme value,
the standup ability is at the second level if the buttocks contact the surface at the first timing and do not contact the surface at a second timing when the center-of-gravity acceleration reaches the second extreme value, and
the standup ability is at the third level if the buttocks contact the surface at the first timing and the second timing.

6. The apparatus according to claim 1, wherein the determination unit classifies the standup ability into a first level, a second level, a third level and a fourth level, the first level being a level at which the subject is able to stand up, the second level being a level at which the subject is able to stand up more slowly than at the first level, the third level being a level at which the subject is able to have the buttocks leave the surface but is unable to stand up, the fourth level being a level at which the subject is unable to stand up.

7. The apparatus according to claim 6, wherein the determination unit determines that:
the standup ability is at the first level if the buttocks do not contact the surface at a first timing when the center-of-gravity acceleration reaches the first extreme value,
the standup ability is at the second level if the buttocks contact the surface at the first timing and do not contact the surface at a second timing when the center-of-gravity acceleration reaches the second extreme value and do not contact the surface within a given time from the second timing,
the standup ability is at the third level if the buttocks contact the surface at the first timing and do not contact the surface at the second timing and contact the surface again within the given time from the second timing, and
the standup ability is at the fourth level if the buttocks contact at the first timing and the second timing.

8. The apparatus according to claim 1, further comprising an output unit configured to output information relating to the standup ability of the subject.

9. The apparatus according to claim 1, further comprising an assistance output unit configured to generate an output to help the subject to stand up, in accordance with levels of the standup ability.

10. The apparatus according to claim 9, wherein the assistance output unit sets the output to inversely proportional to the standup ability.

11. The apparatus according to claim 10, wherein the assistance output unit comprises a motor, and amplitude of the output is amplitude of a torque of the motor.

12. A standup assistance method, comprising:
measuring a center-of-gravity acceleration at which a position of a center-of-gravity of a subject moves;
detecting whether or not buttocks of the subject contact a surface;
determining a standup ability of the subject in accordance with whether or not the buttocks contact the surface when the center-of-gravity acceleration reaches a first extreme value or a second extreme value which is a next extreme value of the first extreme value.

13. The method according to claim 12, wherein the center-of-gravity acceleration is acceleration at which the position of the center-of-gravity moves in a vertical direction, the first extreme value is a maximum value of the center-of-gravity acceleration, and the second extreme value is a minimum value of the center-of-gravity acceleration, if the center-of-gravity acceleration is regarded as increasing upward in the vertical direction.

14. The method according to claim 12, wherein the center-of-gravity acceleration is amplitude of acceleration of the position of the center-of-gravity, and the first extreme value and the second extreme value are maximum values of the center-of-gravity acceleration if the center-of-gravity acceleration is regarded as increasing upward in the vertical direction.

15. The method according to claim 12, wherein the determining the standup ability classifies the standup ability into a first level, a second level and a third level, the first level being a level at which the subject is able to stand up, the second level being a level lower than the first level and higher than a third level, the third level being a level at which the subject is unable to stand up.

16. The method according to claim 15, wherein the determining the standup ability determines that;
the standup ability is at the first level if the buttocks do not contact the surface at a first timing when the center-of-gravity acceleration reaches the first extreme value,
the standup ability is at the second level if the buttocks contact the surface at the first timing and do not contact the surface at a second timing when the center-of-gravity acceleration reaches the second extreme value, and
the standup ability is at the third level if the buttocks contact the surface at the first timing and the second timing.

17. The method according to claim 12, wherein the determining the standup ability classifies the standup ability into a first level, a second level, a third level and a fourth level, the first level being a level at which the subject is able to stand up, the second level being a level at which the subject is able to stand up more slowly than at the first level, the third level being a level at which the subject is able to have the buttocks leave the surface but is unable to stand up, the fourth level being a level at which the subject is unable to stand up.

18. The method according to claim 17, wherein the determining the standup ability determines that:
the standup ability is at the first level if the buttocks do not contact the surface at a first timing when the center-of-gravity acceleration reaches the first extreme value,
the standup ability is at the second level if the buttocks contact the surface at the first timing and do not contact the surface at a second timing when the center-of-gravity acceleration reaches the second extreme value and do not contact the surface within a given time from the second timing,
the standup ability is at the third level if the buttocks contact the surface at the first timing and do not contact the surface at the second timing and contact the surface again within the given time from the second timing, and
the standup ability is at the fourth level if the buttocks contact at the first timing and the second timing.

19. The method according to claim 12, further comprising outputting information relating to the standup ability of the subject.

20. The method according to claim 12, further comprising generating an output to help the subject to stand up, in accordance with levels of the standup ability.
